(19) United States
(12) Patent Application Publication
(10) Pub. No.: US 2016/0068453 A1
(43) Pub. Date: Mar. 10, 2016

(54) METHODS FOR PRODUCING NORMAL PARAFFIN FROM A RENEWABLE FEEDSTOCK

(71) Applicant: UOP LLC, Des Plaines, IL (US)

(72) Inventors: Geoffrey William Fichtl, Chicago, IL (US); Daniel Ellig, Arlington, IL (US)

(21) Appl. No.: 14/482,919

(22) Filed: Sep. 10, 2014

(51) Int. Cl. C07C 1/22; C07C 2/70; C07C 5/333

(52) U.S. Cl. CPC C07C 1/22; C07C 5/3337; C07C 2/70; C07C 2523/42; C07C 2527/051; C07C 2527/045

(57) ABSTRACT

Methods are provided for producing normal paraffins. A method includes contacting a feedstock with a deoxygenation catalyst to produce a paraffin stream, where the feedstock includes a natural oil, and where the deoxygenation catalyst is sulfided. The reactions conditions are controlled when the feedstock contacts the deoxygenation catalyst to control a C11 to C12 normal paraffin ratio, by weight to within about 0.4 to about 1.7.

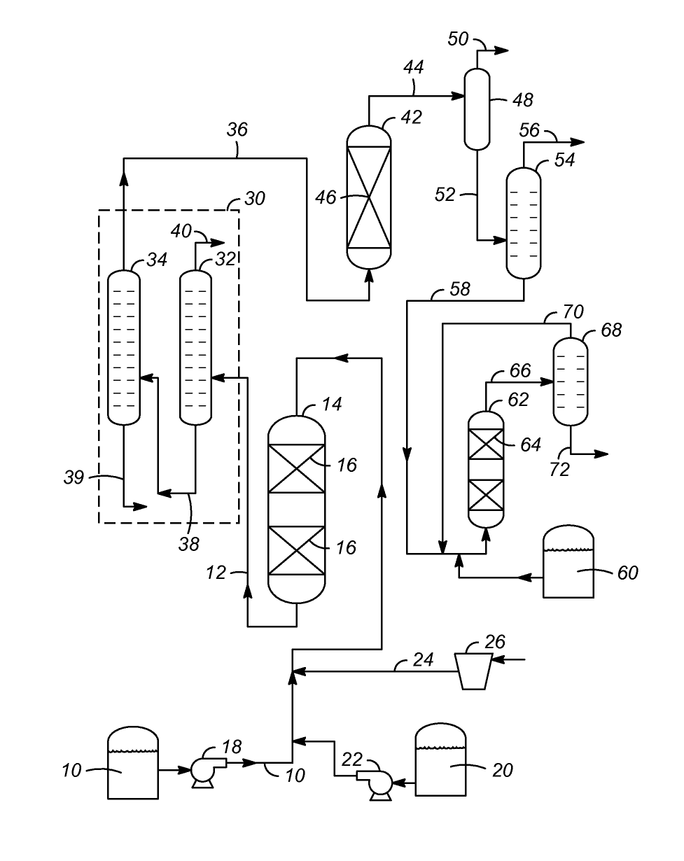

### TECHNICAL FIELD

The present disclosure generally relates to methods for producing normal paraffins from renewable feedstocks, and more particularly relates to methods for converting renewable feedstocks into normal paraffins with a desired ratio of C11 to C12 normal paraffins.

### BACKGROUND

Many detergents include linear alkyl benzenes to facilitate the cleaning process. Linear paraffins are one raw material that can be used in the production of linear alkyl benzenes, but detergent producers prefer specific lengths for the alkyl component of the linear alkyl benzenes. Kerosene boiling range normal alkyls have been used to produce linear alkyl benzenes, where the kerosene is a petroleum product. However, petroleum is a non-renewable resource, so kerosene from petroleum is also a non-renewable resource. There are also legal and social pressures to utilize more renewable resources for consumer products, including detergents.

Normal alkyl paraffins can be produced from natural oils, but most natural oils provide normal alkyl paraffins that are too long (i.e., include too many carbon atoms) to satisfy the detergent producers specifications. Natural oils include fatty acids and triglycerides that can be converted to normal paraffins, but the fatty acids and triglycerides almost exclusively include paraffins with an even number of carbon atoms. The detergent producers' specifications include a mixture of even and odd numbered normal paraffins, where the even and odd numbered normal paraffins fall within desired concentration ranges. Different reaction mechanisms can be used to convert natural oils to normal paraffins, but each reaction mechanism produces either an even numbered normal paraffin or an odd numbered normal paraffin. Processes are typically operated such that one type of reaction mechanism is favored, so the resulting product generally includes a majority of normal paraffins with an even number of carbon atoms or an odd number of carbon atoms. Therefore, existing processes tend to produce normal paraffins that do not have the desired concentration of both normal paraffins with an even number of carbon atoms and an odd number of carbon atoms to satisfy linear alkyl benzene producers' desired concentration ranges.

Accordingly, it is desirable to develop methods for controlling the ratio of normal paraffins produced from natural oils that have an odd number of carbon atoms and an even number of carbon atoms. In addition, it is desirable to develop methods for converting natural oils to normal paraffins with the desired ratio of odd and even carbon atoms while limiting side reactions that decrease the yield of the normal paraffins. Furthermore, other desirable features and characteristics of the present embodiment will become apparent from the subsequent detailed description and the appended claims, taken in conjunction with the accompanying drawings and this background.

### BRIEF SUMMARY

Methods are provided for producing a normal paraffin. A method includes contacting a feedstock with a deoxygenation catalyst to produce a paraffin stream, where the feedstock includes a natural oil, and where the deoxygenation catalyst is sulfided. The reaction conditions are controlled when the feedstock contacts the deoxygenation catalyst to control a C11 to C12 normal paraffin ratio by weight to within about 0.4 to about 1.7.

Another method is provided for producing normal paraffins. A feedstock is contacted with a deoxygenation catalyst in the presence of hydrogen to produce a paraffin stream, where the feedstock includes a natural oil, and where the deoxygenation catalyst is sulfided. A C11 to C12 normal paraffin ratio of the paraffin stream is controlled to within a desired range by controlling a reaction condition. The paraffin stream is fractionated to produce a fractionation effluent including a C10 paraffin, a C11 paraffin, a C12 paraffin, and a C13 paraffin. The fractionation effluent includes about 5 to about 15 weight percent of the C10 paraffin, about 28 to about 45 weight percent of the C11 paraffin, about 28 to about 40 weight percent of the C12 paraffin, and about 10 to about 30 weight percent of the C13 paraffin.

Yet another method is also provided for producing normal paraffins. The method includes selecting a feedstock comprising about 80 weight percent or more glycerides or fatty acids, where the glycerides or fatty acids comprise lauric acid as a component at about 40 weight percent or greater. The feedstock is contacted with a deoxygenation catalyst in the presence of hydrogen, where the deoxygenation catalyst is sulfided, and a C11 to C12 normal paraffin ratio is controlled to within a desired range.

### BRIEF DESCRIPTION OF THE DRAWINGS

Various embodiments will hereinafter be described in conjunction with the FIGURE, which is a schematic diagram of an exemplary embodiment of an apparatus and method for producing normal paraffins from a natural feedstock, and for reacting the normal paraffins with benzene to produce linear alkyl benzenes.

### DETAILED DESCRIPTION

The following detailed description is merely exemplary in nature and is not intended to limit the application or uses of the embodiment described. Furthermore, there is no intention to be bound by any theory presented in the preceding technical field, background, brief summary, or the following detailed description.

Fatty acids and triglycerides in natural oils can be converted to normal paraffins by hydrodeoxygenation, decarboxylation, or decarbonylation. The hydrodeoxygenation process preserves the carbon atoms present in the fatty acid or triglyceride, while the three carbon atoms from the triglyceride backbone are removed. Therefore, hydrodeoxygenation produces normal paraffins having an even number of carbon atoms because natural oils almost exclusively produce fatty acid or triglyceride groups with an even number of carbon atoms, as mentioned above. The decarboxylation and decarbonylation processes convert one of the carbon atoms of the fatty acid or triglyceride into a carbon dioxide or carbon monoxide compound, respectively, so the resulting normal paraffin has an odd number of carbon atoms. The ratio of the hydrodeoxygenation reaction to the decarboxylation/decarbonylation reactions can be determined by measuring the ratio of normal paraffins having odd and even numbers of carbon atoms, where the odd number is one less than the even number (because the extra carbon was lost to carbon oxides). The hydrodeoxygenation reaction, the decarboxylation reaction, and the decarbonylation reaction are collectively referred to as deoxygenation, and all three reactions can occur when a natural oil is contacted with a deoxygenation catalyst at deoxygenation reaction conditions. The normal paraffin ratio of odd to even numbered carbon atoms can be adjusted by controlling several different reaction conditions. For example, the ratio can be shifted towards more odd numbered normal paraffins by increasing a sulfiding agent injection rate for a sulfided deoxygenation catalyst, increasing the liquid hourly space velocity, decreasing the hydrogen feed rate relative to the natural feedstock feed rate (the hydrogen to feedstock ratio), decreasing the reaction pressure, increasing the reaction temperature, or combinations of the above. Therefore, the ratio of normal paraffins having an odd number of carbon atoms to an even number of carbon atoms can be adjusted to within a desired range by controlling the various reaction conditions. The resulting normal paraffin stream can then be used to produce linear alkyl benzenes for detergents, or it can be otherwise used.

Reference is made to the exemplary embodiment illustrated in the FIGURE. A feedstock 10 is deoxygenated to produce a paraffin stream 12. As used herein, “Deoxygenation” means hydrodeoxygenation, decarboxylation, decarbonylation, or a mixture thereof. The feedstock 10 is selected to include a natural oil, and in exemplary embodiments the feedstock 10 includes about 30 weight percent or more natural oil, about 50 weight percent or more natural oil, or about 80 weight percent or more natural oil, where the maximum weight percent of natural oil in the feedstock 10 is 100.

Natural oils include high concentrations of fatty acids and/or triglycerides, where triglycerides are formed by three fatty acid molecules that are bonded together with a glycerol bridge. Natural oils may also include other glycerides in lower concentrations, such as monoglycerides and diglycerides, and these are processed the same as the triglycerides and fatty acids. The glycerol molecule includes three hydroxyl groups (HO—) and each fatty acid molecule has a carboxyl group (COOH). In the glycerides, the hydroxyl groups of the glycerol join the carboxyl groups of the fatty acids to form ester bonds. During deoxygenation, the fatty acids are freed from the triglyceride structure and are converted into normal paraffins. The glycerol backbone is converted into propane, and the oxygen in the ester groups of the triglyceride are converted into water, carbon dioxide, or carbon monoxide. Hydrodeoxygenation results in the normal paraffin having the same number of carbon atoms as the fatty acid chains from which they are derived, so the resulting normal paraffin will have an even number of carbon atoms. If the same compound is decarboxylated or decarbonylated, a carbon dioxide or carbon monoxide molecule is produced, respectively, and the normal paraffin will have one less carbon atom than the fatty acid chain from which it is derived, so the resulting normal paraffin will have an odd number of carbon atoms. Decarboxylation produces carbon dioxide, and decarbonylation produces water and carbon monoxide, but each reaction mechanism produces the same normal paraffin.

The natural oil selected for the feedstock 10 can be obtained from a variety of different sources. The natural oil may include about 40 weight percent or more lauric acid as a component, where lauric acid is a fatty acid with 12 carbon atoms. The lauric acid may be in an ester form in triglycerides, but it can still be referred to as lauric acid. Several plants produce natural oils with high concentrations of lauric acid, such as coconut oil, palm kernel oil, or babassu oil. In an exemplary embodiment, the feedstock 10 is 100% coconut oil, palm kernel oil, babassu oil, or a combination thereof, but in alternate embodiments the feedstock 10 includes other natural oils or other co-feeds, as described above. In an alternate embodiment, castor oil (another natural oil) can be processed to produce normal paraffins with 12 carbon atoms. In this description, the capital letter “C” followed by a number indicates the number of carbon atoms in a molecule, so a C12 normal paraffin is a normal paraffin with 12 carbon atoms. Castor oils are primarily C18 fatty acids with an additional hydroxyl group at the carbon 12 position, and the fatty acids from castor oils are called ricinoleic acid. During deoxygenation, it has been found that some of the carbon chains are cleaved at the carbon 12 position, so castor oil can produce C11 or C12 normal paraffins, as well C17 and C18 normal paraffins when the fatty acid is not cleaved at the carbon 12 hydroxyl group. Other natural oils that include additional hydroxyl groups may also exist. It may also be possible to genetically engineer micro-organisms or algae to produce natural oils with high concentrations of lauric acid, and micro-organisms or algae that produce natural oils with high concentrations of lauric acid may be discovered without the need for genetic engineering. It may also be possible to modify regular vegetable oils or other natural oils to contain internal hydroxyl groups, such as soybean, corn oil, or a wide variety of other natural oil. Such modified vegetable oils behave similar to castor oil upon deoxygenation, and therefore may produce economically significant quantities of paraffins in the C10 to C14 range.

It may be desirable to test the feedstock 10 to verify it includes natural oils, which are a bio-based material, and to determine the concentration of natural oils within the feedstock 10. ASTM test method D6866-05, “Determining the Bio-based Content of Natural Range Materials Using Radiocarbon and Isotope Ratio Mass Spectrometry Analysis” measures the ratio of radioactive carbon 14 to non-radioactive carbon 12 (¹⁴C/¹²C isotope ratio.) A sample is tested and compared to the ¹⁴C/¹²C isotope ratio of a standard. Bio-based materials, including natural oils, are organic materials in which the carbon is incorporated into the bio-based material recently, on a geologic time scale. Plants fix carbon dioxide (CO2) in the atmosphere using photosynthesis, and a small amount of the carbon atoms in the atmosphere are radioactive ¹⁴C. Energy from the sun contacts carbon in the atmosphere and creates a background level of ¹⁴C that is incorporated into all living creatures. When a living creature dies, there is no more uptake of ¹⁴C, so the concentration of ¹⁴C begins to decline as it radioactively decays. The half-life of ¹⁴C is about 5,730 years, so bio-based materials retain close to the equilibrium concentration present in living organisms for some time. However, petroleum was formed millions of years ago, so petroleum products have essentially no ¹⁴C. Therefore, the amount of bio-based natural oils in a hydrocarbon feedstock 10 can be determined by comparing the ¹⁴ C/¹²C ratio to the background level. A background level of 100 pMC (percent modern carbon) was established based on the year 1950, but atmospheric nuclear testing has increased the ¹⁴C concentration to a level of about 107.5 pMC today. Therefore, a product can be tested to determine the percent natural oil using the ASTM D6866-05 method. In various embodiments, the feedstock 10 has a pMC of about 32 (which is about 30% bio-based,) or about 54 (which is about 50% bio-based,) or about 86 (which is about 80% bio-based.)

Nitrogen has an inhibitory effect on the deoxygenation process, so the feedstock 10 may include about 5 parts per million nitrogen or less in an exemplary embodiment. “Nitrogen concentrations” discussed herein are the amount of elemental nitrogen in a compound, by weight, and can be measured by various techniques such as chemiluminescence techniques described in UOP Method 981, available from ASTM. Higher reaction temperatures can be used to overcome the inhibitory effect of nitrogen, but the higher temperatures tend to produce more undesired cracking and/or isomerization of the normal paraffins. Purification steps can be taken to reduce the nitrogen concentration, such as refining the oil, as understood by those skilled in the art.

The natural oils in the feedstock 10 may contain a variety of impurities. For example, tall oil is a byproduct of the wood processing industry, and includes esters and rosin acids in addition to free fatty acids. Rosin acids are cyclic carboxylic acids. The natural oils or optional petroleum based co-feeds in the feedstock 10 may also contain contaminants such as alkali metals, (e.g. sodium and potassium), phosphorous, various solids, water, detergents, or other impurities. In some embodiments, the feedstock 10 is pre-cleaned in an optional pre-cleaning zone (not illustrated) to improve downstream processing operations, or the natural oil may optionally be pre-cleaned before being combined with a co-feed in embodiments where the feedstock 10 includes a non-natural oil component. Several different types of pre-cleaning are possible. For example, the pre-cleaning zone may be configured to provide a mild acid wash by contact with dilute sulfuric, nitric, citric, phosphoric, or hydrochloric acid in a reactor. The acid wash can be a continuous process or a batch process, and the dilute acid contact can be at ambient temperature and atmospheric pressure. Other possible pre-cleaning steps for either the natural oil or the feedstock 10 (in embodiments where the feedstock 10 includes co-feeds besides the natural oil) include, but are not limited to, contact with an ion exchange resin such as Amberlyst®-15, a caustic treatment, bleaching, filtration, solvent extraction, hydro processing, pre-treatment with a guard bed, or combinations of the above.

The feedstock 10 is deoxygenated in a deoxygenation unit 14 that contains a deoxygenation catalyst 16. The feedstock 10 is contacted with the deoxygenation catalyst 16 at deoxygenation reaction conditions to produce the paraffin stream 12, where the paraffin stream 12 includes normal paraffins. A feedstock pump 18 may be used to introduce the feedstock 10 to the deoxygenation unit 14, but gravity, pressure, or other methods can be used in alternate embodiments. In an exemplary embodiment, the feedstock 10 is added to the deoxygenation unit 14 at a rate sufficient to produce a liquid hourly space velocity of about 0.2 to about 10 hr⁻¹.

The deoxygenation catalyst 16 is sulfided, and a sulfiding agent 20 is added to the deoxygenation catalyst 16 to maintain it in a sulfided state. The sulfiding agent 20 is introduced to the deoxygenation catalyst 16 before contact with the feedstock 10, and further additions of the sulfiding agent 20 may be used to maintain the deoxygenation catalyst 16 in a sulfided state. Continuing additions of the sulfiding agent 20 may be combined with the feedstock 10 before contact with the deoxygenation catalyst 16, or it may be added directly to the deoxygenation catalyst 16. A sulfide agent pump 22 can be used to introduce the sulfiding agent 20. Pressure, gravity, or other methods can be used in place of the sulfide agent pump 22 in various embodiments. The sulfur added to the deoxygenation catalyst 16 is measured as elemental sulfur, regardless of the sulfiding agent 20 containing the sulfur, and a wide variety of sulfiding agents 20 can be used. For example, suitable sulfiding agents 20 include, but are not limited to, dimethyl disulfide, tertiary butyl sulfide, dibutyl disulfide, and hydrogen sulfide. The sulfur may be obtained from various sources, such as part of a hydrogen stream 24 (described below), as part of the feedstock 10, or as a separate sulfiding agent 20 as illustrated. Therefore, a separate sulfiding agent introduction system may not be needed in some embodiments, such as embodiments where the feedstock 10 or hydrogen stream 24 include sufficient sulfur for the deoxygenation catalyst 16. Sulfur concentrations of about 5,000 ppm or less, such as about 5,000 ppm to about 100 ppm, are typically sufficient to maintain the deoxygenation catalyst 16 in a sulfided state.

The feedstock 10 is contacted with the deoxygenation catalyst 16 in the presence of hydrogen, and hydrogen is provided by a hydrogen stream 24 in an exemplary embodiment. A hydrogen compressor 26 may be used to introduce the hydrogen stream 24 to the deoxygenation unit 14, but pressurized containers or other methods can be used in alternate embodiments. Hydrogen from the hydrogen stream 24 may be added at a ratio of about 2.7 standard cubic meters of hydrogen per liter of feedstock or less, where a standard cubic meter is measured at 15.6° C. and 1 atmosphere of pressure. The hydrodeoxygenation reaction consumes hydrogen and produces water as a byproduct, while the decarbonylation and decarboxylation reactions produce carbon monoxide (CO) or carbon dioxide (CO₂), respectively, while consuming less hydrogen than hydrodeoxygenation. Hydrogen is present for all the reactions in the deoxygenation unit 14, regardless of the mechanism of deoxygenation.

The paraffin stream 12 produced in the deoxygenation unit 14 includes a liquid portion and a gaseous portion. The liquid portion includes hydrocarbon compounds that are largely normal paraffin compounds (n-paraffins). The gaseous portion includes hydrogen, carbon dioxide (CO₂), carbon monoxide (CO), water vapor, propane, and perhaps sulfur components such as hydrogen sulfide. The hydrogen and other gases may be separated from the liquid portion in a variety of manners, such as a fractionation or a gas separator (not illustrated), and the hydrogen may be recovered and re-used in some embodiments.

The deoxygenation unit 14 includes the deoxygenation catalyst 16, which is in a sulfided state. Conventional deoxygenation catalysts 16 may be used, such as those including one or more of nickel (Ni), molybdenum (Mo), Cobalt (Co), or Phosphorus (P) on high surface area supports such as aluminas, silica, titania, zirconia, and mixtures thereof. Other deoxygenation catalysts 16 include one or more noble metal catalytic elements dispersed on a high surface area support. Non-limiting examples of noble metals include platinum (Pt) and/or palladium (Pd). Deoxygenation reaction conditions include a reaction temperature of about 250 degrees centigrade (° C.) to about 400° C., and a reaction pressure of about 1,700 kilopascals (kPa) absolute to about 5,500 kPa absolute. Other reaction conditions for the deoxygenation unit 14 can also be used.

The deoxygenation unit 14 can crack and/or isomerize the normal paraffins in the paraffin stream 12 if the reaction conditions are too severe. Any cracking or isomerization reduces the yield of normal paraffins, so the reaction conditions in the deoxygenation unit 14 can be controlled to minimize the cracking and/or isomerization of the normal paraffins. For example, the reaction temperature may be limited to about 400° C. or less, such as from about 400° C. to about 250° C. Cracking and/or isomerization of the normal paraffins can also be minimized by selecting an appropriate deoxygenation catalyst 16, where a less active deoxygenation catalyst 16 is less likely to crack or isomerize the normal paraffins.

Natural oils with fatty acids or esters having 10 carbon atoms (C10 fatty acids or esters) produce either C9 or C10 normal paraffins in the deoxygenation process. In this description, the capital letter “C” followed by a number indicates the number of carbon atoms in a molecule, so a C10 normal paraffin is a normal paraffin with 10 carbon atoms. Natural oils with C14 fatty acids or esters produce either C13 or C14 normal paraffins. In an exemplary embodiment, the paraffin stream 12 is further processed to include normal paraffins with 10, 11, 12, or 13 carbon atoms, where normal paraffins with 9 or fewer carbon atoms or 14 or more carbon atoms are separated. The C9 and smaller normal paraffins and the C14 and larger normal paraffins can be separated from the C10, C11, C12, and C13 normal paraffins by fractionation in a fractionation zone 30 to produce a fractionation effluent 36. In the embodiment illustrated, the fractionation zone 30 includes a first paraffin fractionator 32 and a second paraffin fractionator 34, but the fractionation zone 30 may include more or fewer fractionators in alternate embodiments. Since the C10 and C14 normal paraffins can be separated from the paraffin stream 12 to produce the fractionation effluent 36, the ratio of C13 to C14 normal paraffins is not critical, and the ratio of C9 to C10 normal paraffins is not critical. All ratios of one paraffin to another in this description are weight/weight ratios, unless otherwise specified. Essentially all of the C12 and C11 paraffins from the paraffin stream 12 pass through the fractionation zone 30 into the fractionation effluent 36, so the C11 to C12 normal paraffin ratio in the paraffin stream 12 should be controlled. In an exemplary embodiment, the desired concentration of C11 paraffins in the fractionation effluent 36 is about 28 to about 45 weight percent, and the desired concentration of C12 paraffins is about 28 to 40 weight percent, so the desired ratio of C11 to C12 normal paraffins is about 28/40 to 45/28, or about 0.7 to about 1.7. In an alternate embodiment, the C11 to C12 normal paraffin ratio is controlled to within about 0.4 to about 1.7 in an embodiment where a different ratio of C11 to C12 normal paraffins is desired. In yet other embodiments, the C11 to C12 normal paraffin ratio is controlled to within about 0.6 to about 1.0, or within from about 0.8 to about 1.5, or within from about 0.8 to about 1.2. The normal paraffins in the paraffin stream 12 are passed through the fractionation zone 30 to the fractionation effluent 36, so the C11 to C12 normal paraffin ratio in the paraffin stream 12 is about the same as the C11 to C12 normal paraffin ratio in the fractionation effluent 36.

The ratio of the normal paraffins with an odd number of carbon atoms to the normal paraffins with an even number of carbon atoms can be controlled by adjusting various reaction conditions. In this description, the C11 to C12 ratio is used to exemplify the ratio of normal paraffin with an odd number of carbon atoms to the normal paraffins with an even number of carbon atoms, but it should be understood that other ratios could be used instead, such as the C9 to C10 ratio, C13 to C14 ratio, etc. This ratio can also be controlled by utilizing different oxygenation catalysts 16, but reaction conditions can be changed more easily and rapidly than catalysts. It has been found that the C11 to C12 ratio is increased by each of the following reaction conditions: A) increase the sulfur feed to the deoxygenation catalyst 16; B) increase the liquid hourly space velocity of the feedstock 10 to the deoxygenation unit 14; C) decrease a hydrogen to feedstock 10 ratio, which is the hydrogen feed rate divided by the feedstock feed rate; D) decrease the reaction pressure in the deoxygenation unit 14; or E) increase the reaction temperature in the deoxygenation unit 14. Each of these 5 reaction conditions can be adjusted individually or in any combination to control and adjust the C11 to C12 normal paraffin ratio. The examples described below demonstrate these effects. Changing the reaction conditions also adjusts the ratio of other normal paraffin pairs (such as the C13 to C14 normal paraffin ratio, the C15 to C16 normal paraffin ratio, the C9 to C10 normal paraffin ratio, etc.), but modifications to other normal paraffin pairs is not critical to obtaining the proper component concentrations in the fractionation effluent 36. In an exemplary embodiment, the C11 to C12 normal paraffin ratio is controlled by controlling a subset of the listed reaction conditions, such as the sulfur injection rate, the liquid hourly space velocity, and/or the reaction pressure, while the other reaction conditions are maintained. Some of the reaction conditions may be easier to control, have fewer side effects, or provide more consistent or better control of the C11 to C12 normal paraffin ratio, and the reaction conditions that are most effective may vary from one process to the next.

Once the C11 to C12 normal paraffin ratio is controlled, the concentration of C10 and C14 normal paraffins can be adjusted in the fractionation zone 30 by removing excess C10 or C14, as desired. As such, the concentration of the various components of the fractionation effluent 36 can be controlled by adjusting the reaction conditions in the deoxygenation unit 14, and by controlling the fractionation in the fractionation zone 30.

The fractionation zone 30 produces the fractionation effluent 36, which includes C10 to C14 normal paraffins in some embodiments, but it also produces one or more deoxygenation heavy ends streams, such as a first deoxygenation heavy ends stream 38 and a second deoxygenation heavy ends stream 39. The fractionation zone 30 may also produce one or more deoxygenation light ends stream 40. The paraffin stream 12 may pass through a separator (not illustrated) before entering the fractionation zone 30 to separate excess hydrogen and other byproducts, and the excess hydrogen may be recovered and reused or transferred to other processes. The deoxygenation heavy ends stream 38 includes compounds with a boiling point higher than the C14 normal paraffins, (about 254° C. at atmospheric pressure) and this stream can be further processed and used, such as for fuel or other purposes. The deoxygenation light ends stream 40 includes compounds with a boiling point lower than that of the C10 normal paraffins, (about 174° C. at atmospheric pressure) and this stream can be further processed and used, such as for a fuel or other uses.

The normal paraffins in the fractionation effluent 36 should have a mixture of normal paraffins with different numbers of carbon atoms, with a specific range of acceptable weight percentages for the normal paraffins for each number of carbon atoms. In an exemplary embodiment, the normal paraffins should include about 5 to about 15 weight percent C10 normal paraffins, about 28 to about 45 weight percent C11 normal paraffins, about 28 to about 40 weight percent C12 normal paraffins, and about 10 to about 30 weight percent C13 normal paraffins. There should be about 5 weight percent or less C9 or smaller normal paraffins, and about 5 weight percent or less C14 or larger normal paraffins. However, in other embodiments, different concentration ranges may be desired, and the different concentration ranges may include the same or different carbon chain lengths for the normal paraffins. The normal paraffins are produced within specified concentration ranges for different uses, so the specified concentration ranges can vary for different uses. The concentration ranges listed above are an example of the desired concentration ranges for normal paraffins that are eventually incorporated into detergent products, but other detergent products or other types of products may have different desired concentration ranges.

The fractionation effluent 36 can be further processed in some embodiments. In an exemplary embodiment, the fractionation effluent 36 is introduced into a dehydrogenation unit 42 to produce a mono-olefin stream 44 comprising mono-olefins. The dehydrogenation unit 42 dehydrogenates the normal paraffins into mono-olefins having the same carbon number as the normal paraffin. Typically the dehydrogenation unit 42 uses a dehydrogenation catalyst 46, as understood by those skilled in the art. The dehydrogenation unit 42 may also produce some diolefins and aromatics. In an exemplary embodiment, the dehydrogenation catalyst 46 is platinum on alumina catalyst where the platinum is attenuated with an attenuator metal. Reaction conditions for the dehydrogenation unit 42 include liquid hourly space velocities from about 5 to about 50 hr⁻¹, pressures from about 30 to about 400 kPa, and temperatures from about 400° C. to about 500° C. The hydrogen to hydrocarbon mole ratio may be from about 1 to about 12. Dehydrogenation of normal paraffins is an equilibrium-limited process that limits conversion of paraffins to olefins, so the mono-olefin stream 44 will also include unreacted normal paraffins. In some embodiments, about 12 weight percent of the normal paraffins are converted to mono-olefins in the dehydrogenation unit 42, so the concentration of normal paraffins in the mono-olefin stream 44 can be significant. The unreacted normal paraffins may be separated at a later stage in the process, and can then be recycled back to the dehydrogenation unit 42 or otherwise used. This recycle stream is not illustrated for simplicity and clarity.

The mono-olefin stream 44 may then pass to a dehydrogenation phase separator 48 to remove hydrogen in a dehydrogenation phase separator hydrogen stream 50, and also produce a dehydrogenation liquid stream 52. The dehydrogenation liquid stream 52 includes the mono-olefins, the di-olefins, and the aromatics. The dehydrogenation liquid stream 52 may optionally be passed through a hydrogenation unit (not illustrated) to hydrogenate at least some of the diolefins into mono-olefins. The dehydrogenation liquid stream 52 may flow into a lights separator 54, such as a stripper column, where a dehydrogenation lights stream 56 is removed to leave a dehydrogenation heavy ends stream 58. The dehydrogenation lights stream 56 includes butane, propane, ethane, and methane that may have formed in the dehydrogenation unit 42 or other upstream processes, and the dehydrogenation heavy ends stream 58 includes the mono-olefins, any remaining diolefins, and aromatics. The aromatics may optionally be removed in an aromatic removal unit (not illustrated), as understood by those skilled in the art.

The dehydrogenation heavy ends stream 58 may be alkylated with benzene 60 in an alkylation unit 62. The alkylation unit 62 holds an alkylation catalyst 64, such as a solid acid catalyst, that facilitates alkylation of the benzene 60 with the mono-olefins in the dehydrogenation heavy ends stream 58. Exemplary embodiments of the alkylation catalyst 64 include fluorided silica-alumina, hydrogen fluoride, aluminum chloride, and zeolitic catalysts. The alkylation unit 62 produces an alkylation effluent stream 66 that includes linear alkylbenzenes, as well as small amounts of hydrogen and low boiling hydrocarbons, such as those boiling below about 10° C. at atmospheric pressure. Suitable reaction conditions for the alkylation unit 62 include liquid hourly space velocities from about 1 to about 10 hr⁻¹, pressures to maintain liquid phase operation such as about 2,000 kPa to about 5,000 kPa, temperatures from about 80° C. to about 200° C., and benzene to olefin mole ratios of from about 3 to about 40.

Surplus benzene 60 is supplied to the alkylation unit 62 for a high degree of alkylation. Therefore, the alkylation effluent stream 66 may be introduced to a benzene stripper 68 to recover excess benzene 60 in a benzene stripper light ends stream 70. The benzene stripper light ends stream 70 can be added back to the alkylation unit 62 or otherwise used. An alkylbenzene stream 72 exits the benzene stripper 68, where the alkylbenzene stream 72 includes the linear alkylbenzenes produced in the alkylation unit 62, and possibly other compounds such as n-paraffins, unalkylated mono-olefins, and other compounds. The linear alkylbenzene production system described above may include additional units in various embodiments to provide an alkylbenzene stream 72 with suitable composition and purity.

### EXAMPLE 1

Coconut oil was introduced to a test catalyst at a reaction temperature of 316° C., a reaction pressure of 3,309 kPa, and a hydrogen to hydrocarbon feed ratio of 7,200 standard cubic feet per barrel (1.3 standard cubic meters per liter). The sulfur feed was changed (measured in parts per million by weight (ppmw)), and the mole/mole ratio of C11 to C12 normal paraffins (C11/12 ratio) was measured. A sulfur feed rate of 500 produced a C11/12 ratio of 0.4, a sulfur feed rate of 1,000 produced a C11/12 ratio of 0.3 to 0.4, a sulfur feed rate of 1,500 produced a C11/12 ratio 0.6 to 0.8, a sulfur feed rate of 2,500 produced a C11/12 ratio of 0.86, and a sulfur feed rate of 3,500 produced a C11/12 ratio of 1.1.

### EXAMPLE 2

Coconut oil was introduced to a test catalyst at a reaction temperature of 312° C., a reaction pressure of 3,309 kPa, and a hydrogen to hydrocarbon feed ratio of 7,600 standard cubic feet per barrel (1.3 standard cubic meters per liter). The sulfur feed was changed (measured in ppmw), and the C11/12 ratio was measured. A sulfur feed rate of 500 produced a C11/12 ratio of 0.21, a sulfur feed rate of 1,500 produced a C11/12 ratio 0.6 to 0.31, a sulfur feed rate of 2,400 produced a C11/12 ratio of 0.35, and a sulfur feed rate of 3,300 produced a C11/12 ratio of 0.38.

### EXAMPLE 3

Coconut oil was introduced to a test catalyst at a reaction temperature of 312° C., a reaction pressure of 3,309 kPa, and a sulfur feed rate of about 1,500 ppmw. The liquid hourly space velocity (LHSV, measured in hr⁻¹) was changed, and the C11/C12 ratio was measured for different liquid hourly space velocities. A LHSV of 0.5 produced a C11/C12 ratio of 0.63. A LHSV of 0.52 produced a C11/C12 ratio of 0.66. A LHSV of 0.78 produced a C11/C12 ratio of 0.69 to 0.70. A LHSV of 1.0 produced a C11/C12 ratio of 0.8.

### EXAMPLE 4

Coconut oil was introduced to a test catalyst at a reaction temperature of 312° C., a sulfur feed rate of 1,500 ppmw, and a reaction pressure of 3,309 kPa. The hydrogen to hydrocarbon feed ratio (H/HC), also referred to as the hydrogen to feedstock ratio, was changed (H/HC measured in standard cubic meters per liter), and the C11/C12 ratio was measured for different H/HC. An H/HC of 1.4 produced a C11/C12 ratio of 0.8. An H/HC of 2.0 produced a C11/C12 ratio of 0.62. An H/HC of 2.1 produced a C11/C12 ratio of 0.62.

### EXAMPLE 5

Coconut oil was introduced to a test catalyst at a reaction temperature of 312° C., a sulfur feed rate of 1,500 ppmw, and an H/HC of 1.4. The reaction pressure (P) was changed (measured in kPa), and the C11/C12 ratio was measured for different reaction pressures. A P of 2758 produced a C11/C12 ratio of 0.9 to 0.92. A P of 3,309 produced a C11/C12 ratio of 0.79 to 0.81.

### EXAMPLE 6

Coconut oil was introduced to a different test catalyst than in Example 5 at a reaction temperature of 312° C., a sulfur feed rate of 1,500 ppmw, and an H/HC of 1.4. The reaction pressure (P) was changed (measured in kPa), and the C11/C12 ratio was measured for different reaction pressures. A P of 2758 produced a C11/C12 ratio of 0.34 to 0.35. A P of 3,309 produced a C11/C12 ratio of 0.30 to 0.31.

### EXAMPLE 7

Coconut oil was introduced to a test catalyst at a reaction pressure of 3309 kPa, a sulfur feed rate of 537 ppmw, and an H/HC of 1.4. The reaction temperate (T) was changed (measured in ° C.), and the C11/C12 ratio was measured for different reaction temperatures. A T of 294 produced a C11/C12 ratio of 0.60 to 0.74. A T of 305 produced a C11/C12 ratio of 0.69 to 0.74. A T of 317 produced a C11/C12 ratio of 0.76 to 0.80.

### EXAMPLE 8

Coconut oil was introduced to a different test catalyst than in Example 7 at a reaction pressure of 3309 kPa, a sulfur feed rate of 1,500 ppmw, and an H/HC of 1.4. The reaction temperate (T) was changed (measured in ° C.), and the C11/C12 ratio was measured for different reaction temperatures. A T of 289 produced a C11/C12 ratio of 0.66 to 0.72. A T of 301 produced a C11/C12 ratio of 0.75 to 0.80. A T of 312 produced a C11/C12 ratio of 0.79 to 0.81.

### EXAMPLE 9

Coconut oil was introduced to a different test catalyst than in Examples 7 and 8 at a reaction pressure of 3309 kPa, a sulfur feed rate of 1,500 ppmw, and an H/HC of 1.4. The reaction temperate (T) was changed (measured in ° C.), and the C11/C12 ratio was measured for different reaction temperatures. A T of 278 produced a C11/C12 ratio of 0.24. A T of 290 produced a C11/C12 ratio of 0.24. A T of 301 produced a C11/C12 ratio of 0.25 to 0.26. AT of 312 produced a C11/C12 ratio of 0.30 to 0.31.

It should be appreciated that the embodiment or embodiments illustrated are only examples, and are not intended to limit the scope, applicability, or configuration of the application in any way. Rather, the foregoing detailed description will provide those skilled in the art with a convenient road map for implementing one or more embodiments, it being understood that various changes may be made in the function and arrangement of elements described without departing from the scope as set forth in the appended claims.

1. A method of producing normal paraffins, the method comprising the steps of:
contacting a feedstock with a deoxygenation catalyst to produce a paraffin stream, wherein the feedstock comprises a natural oil, and wherein the deoxygenation catalyst is sulfided; and
controlling reaction conditions when the feedstock contacts the deoxygenation catalyst to control a C11 to C12 normal paraffin ratio by weight to within about 0.4 to about 1.7.

2. The method of claim 1 wherein contacting the feedstock with the deoxygenation catalyst comprises contacting the feedstock with the deoxygenation catalyst at a temperature of about 400 degrees centigrade or less to minimize cracking of the normal paraffins in the paraffin stream.

3. The method of claim 1 wherein controlling the reaction conditions when the feedstock contacts the deoxygenation catalyst comprises controlling a sulfur injection rate, a reaction temperature, a reaction pressure, a hydrogen to feedstock feed ratio, a liquid hourly space velocity, or a combination thereof.

4. The method of claim 1 wherein controlling the reaction conditions when the feedstock contacts the deoxygenation catalyst comprises controlling a sulfur injection rate, a reaction pressure, a liquid hourly space velocity, or a combination thereof.

5. The method of claim 1 further comprising:
obtaining the feedstock, wherein the feedstock comprises about 5 parts per million by weight elemental nitrogen or less.

6. The method of claim 1 wherein contacting the feedstock with the deoxygenation catalyst comprises contacting the feedstock with the deoxygenation catalyst, wherein the feedstock comprises about 54 percent modern carbon (pMC) or greater, such that the feedstock is about 50 weight percent bio-based or greater.

7. The method of claim 1 further comprising:
obtaining the feedstock, wherein the feedstock comprises coconut oil, palm kernel oil, babassu oil, or a combination thereof at a concentration of about 50 weight percent or greater.

8. The method of claim 1 further comprising:
dehydrogenating the paraffin stream to produce a mono-olefin stream comprising mono-olefins; and
alkylating benzene with the mono-olefin stream to produce an alkylbenzene stream comprising linear alkylbenzenes.

9. The method of claim 1 further comprising:
fractionating the paraffin stream to produce a fractionation effluent comprising a C10 paraffin, a C11 paraffin, a C12 paraffin, and a C13 paraffin, wherein the fractionation effluent comprises from about 5 to about 15 weight percent of the C10 paraffin, from about 28 to about 45 weight percent of the C11 paraffin, from about 28 to about 40 weight percent of the C12 paraffin, and from about 10 to about 30 weight percent of the C13 paraffin.

10. The method of claim 1 further comprising:
obtaining the feedstock, wherein the feedstock comprises about 50 mass percent castor oil, algal oil, microbial oil, modified vegetable oil that behaves similar to castor oil upon deoxygenation, or a combination thereof.

11. A method of producing normal paraffins, the method comprising the steps of:
contacting a feedstock with a deoxygenation catalyst in the presence of hydrogen to produce a paraffin stream, wherein the feedstock comprises a natural oil, and wherein the deoxygenation catalyst is sulfided;
controlling a C11 to C12 normal paraffin ratio of the paraffin stream to within a desired range by controlling a reaction condition; and
fractionating the paraffin stream to produce a fractionation effluent comprising a C10 paraffin, a C11 paraffin, a C12 paraffin, and a C13 paraffin wherein the fractionation effluent comprises from about 5 to about 15 weight percent of the C10 paraffin, from about 28 to about 45 weight percent of the C11 paraffin, from about 28 to about 40 weight percent of the C12 paraffin, and from about 10 to about 30 weight percent of the C13 paraffin.

12. The method of claim 11 wherein controlling the C11 to C12 normal paraffin ratio comprises varying a decarboxylation and decarbonylation reaction rate relative to a hydrodeoxygenation reaction rate, wherein the hydrodeoxygenation reaction rate is decreased relative to the decarboxylation and decarbonylation reaction rate by increasing a sulfur feed to the deoxygenation catalyst, by increasing a liquid hourly space velocity of the feedstock to the deoxygenation catalyst, by decreasing a hydrogen to feedstock ratio; by decreasing a reaction pressure, by increasing a reaction temperature, or a combination thereof.

13. The method of claim 11 further comprising:
dehydrogenating the fractionation effluent to produce a mono-olefin stream comprising mono-olefins.

14. The method of claim 13 further comprising:
alkylating benzene with the mono-olefin stream to produce an alkylbenzene stream comprising linear alkylbenzenes.

15. The method of claim 11 wherein contacting the feedstock with the deoxygenation catalyst comprises contacting the feedstock with the deoxygenation catalyst wherein the feedstock comprises about 50 mass percent of the natural oil or greater.

16. The method of claim 11 wherein contacting the feedstock with the deoxygenation catalyst comprises contacting the feedstock with the deoxygenation catalyst wherein the feedstock comprises about 80 mass percent of the natural oil or greater.

17. The method of claim 11 further comprising:
pre-cleaning the natural oil prior to contacting the feedstock with the deoxygenation catalyst.

18. The method of claim 11 wherein controlling C11 to C12 normal paraffin ratio, by weight comprises controlling the C11 to C12 normal paraffin ratio, by weight to from about 0.8 to about 1.5.

19. The method of claim 11 wherein controlling C11 to C12 normal paraffin ratio, by weight comprises controlling the C11 to C12 normal paraffin ratio, by weight to from about 0.8 to about 1.2.

20. A method of producing normal paraffins, the method comprising the steps of:
selecting a feedstock comprising about 80 weight percent or greater glycerides or fatty acids, wherein the glycerides or fatty acids comprise lauric acid as a component at about 40 weight percent or greater;
contacting the feedstock with a deoxygenation catalyst in the presence of hydrogen, wherein the deoxygenation catalyst is sulfided; and
controlling a C11 to C12 normal paraffin ratio to within a desired range.
